(19) United States
(12) Patent Application Publication
(10) Pub. No.: US 2012/0053467 A1
(43) Pub. Date: Mar. 1, 2012

(54) METHOD AND APPARATUS FOR VOLUME DETERMINATION

(75) Inventor: Nicholas Betts, Thebarton (AU)

(73) Assignee: SIGNOSTICS LIMITED, Thebarton (AU)

(21) Appl. No.: 13/217,274

(22) Filed: Aug. 25, 2011

(30) Foreign Application Priority Data
Aug. 27, 2010 (AU) 2010903842

(51) Int. Cl. A61B 8/00; G06F 3/048

(52) U.S. Cl. 600/449; 715/771

(57) ABSTRACT

A user interface element for a diagnostic ultrasound system including a user determined seed point, a perimeter defining a closed region of similar echoic intensity surrounding the seed point, here that region corresponds to a cross section through a physical feature within a body, a first calculated value being an area of said closed region, a second calculated value being a volume of said physical feature, calculated using preselected assumptions about the shape of the physical feature, wherein the perimeter is user adjustable.

### TECHNICAL FIELD

The present invention relates to a method and apparatus for determining the volume of an organ by use of ultrasound imaging. In particular the method and apparatus may be applicable to determination of the volume of a human or animal bladder.

### BACKGROUND ART

At times it is clinically useful to know the area or spatial volume of a structure within the body, in particular the volume of the bladder. Clinically, the volume of a patient's bladder may be important. It can be used to determine the residual volume of urine in the bladder following voiding, which may be clinically important. It may also be important to determine the amount of urine in the bladder in order to decide when catheterisation is required. Non-invasive bladder volume measurement techniques with ultrasound sonography have been described in the art.

In principle, ultrasound scanning measures distance based on echo travel time. Early echo techniques used a single ultrasound transducer and echo presentation was recorded as echo amplitude versus depth. A method for determining bladder volume to determine residual urine volume based on distance measurement to the dorsal posterior bladder wall was described in the 1960's. The method was not adjusted to specific, shape dependent, measuring configurations.

A relation between the difference in echo travel time between echoes from the posterior and anterior bladder wall and the independently measured bladder volume was recognised. Volume measurement methods based on this observation have been described. The methods are exclusively based on bladder depth measurement. Since the bladder changes in shape when filling, a single distance measurement is not precise enough to predict the entire bladder volume. No bladder shape dependent measurement configuration is used.

Diagnostic ultrasound is today well known for real-time cross-sectional imaging of human organs. For cross-sectional imaging the sound beam is swept electronically or mechanically through the cross section to be imaged. Echoes are presented as intensity modulated dots on a display, giving the well-known ultrasound sector scan display.

A method used in the current art is to perform one or more two-dimensional diagnostic ultrasound ‘B’ scans to produce images of one or more cross sections through the structure whose volume is of interest, such as the bladder, and then to make several standard reference measurements of that imaged structure which are then inserted into a formula to estimate the cross sectional area or volume as required. For the bladder, transverse and sagittal scans are recorded and the height and width of the transverse image and the depth of the longitudinal one are manually measured, then multiplied together to produce a measure of the volume. A scaling constant is usually also included within the calculation which then crudely models the volume of an oblate ellipsoid.

This crude model may have inaccuracies as high as fifty percent. The bladder varies greatly in shape. A single individual's bladder shape will vary according to the degree of filling, most closely approximating the model when significantly full. Between individuals, the shape will vary depending on a number of factors, which may change the actual bladder shape of the apparent shape as shown by an ultrasound scan. The presence or absence of the uterus will change the shape, as will the prostate. Pathology of the bladder, including haematoma, or of the surrounding organs, which may distort the bladder, will also affect the bladder shape.

An ultrasound apparatus for determining the bladder volume is shown in U.S. Pat. No. 4,926,871 to Dipankar Ganguly et al. This discloses a scan head referred to as a sparse linear array with transducers mounted at predetermined angles such that the acoustic “beams” emitted by the transducer tend to a common point. The volume is calculated according to a geometric model. An apparatus is described for automatic calculation of bladder volume from ultrasound measurements in two orthogonal planes. The device is complex, including a stepper motor for deflecting the acoustic “beams”. It requires a skilled operator to manipulate the scan head in a particular way to obtain the ultrasound measurements.

Apparatus exist in the prior art whereby the transducer, and thus the beam, are mechanically swept over the volume of the bladder. Such sweeping takes time, meaning that volume measurement is not available instantaneously. Further, no instantaneous feedback on optimal positioning of the apparatus with respect to the bladder is available. In an exemplary apparatus, bladder volume is measured by interrogating a three-dimensional region containing the bladder and then performing image detection on the ultrasound signals returned from the region insonated. The three dimensional scan is achieved by performing twelve planar scans rotated by mechanically sweeping a transducer through a 97 degree arc in steps of 1.9 degrees. The device is thus mechanically complex and requires complex calculations to yield a result.

Ganguly et al in U.S. Pat. No. 5,964,710 entitled “System for estimating bladder volume” disclose a method for determining bladder volume based on bladder wall contour detection from ultrasound data acquired in a plurality of planes which subdivide the bladder. In each single plane of the plurality of planes N transducers are positioned on a line to produce N ultrasound beams to measure at N positions the distance from front to back wall in the selected plan. From this the surface is derived. This procedure is repeated in the other planes as well. The volume is calculated from the weighted sum of the plurality of planes. In Ganguly's method the entire perimeter of the bladder is echographically sampled in 3 dimensions. The equipment required to undertake this sampling in a clinical context is expensive and complex.

### DISCLOSURE OF THE INVENTION

In one form of this invention there is proposed a user interface element for a diagnostic ultrasound system including a user interface element for a diagnostic ultrasound system adapted to be applied to an ultrasound B-mode image displayed to a user, the interface element including:

a seed point adapted to be positioned by the user to select a seed point on the displayed image;
a perimeter defining a closed region of similar echoic intensity surrounding the seed point, the position of said perimeter being automatically calculated in response to the user positioning the seed point, where said region corresponds to a cross section through a physical feature within a body;
a first calculated value being an area of said closed region;
a second calculated value being a volume of said physical feature calculated using preselected assumptions about the shape of the physical feature.

In preference, the user is able to modify the perimeter placement by dragging a selected point on the perimeter to a different location, the perimeter remaining continuous.

Preferably, this modification of the perimeter includes movement of a deformable portion of the perimeter. This is the portion of the perimeter within a selected radius of the selected point on the perimeter. The deformable portion, which includes the selected point, deforms when the selected point is moved by the user, such that points on the perimeter at the selected radius do not move, while the selected point moves as dragged by the user and points on the perimeter within the deformable portion move by different amounts in such a manner that the perimeter remains continuous.

In a further aspect, the invention lies in a method of calculating the volume of a urinary bladder wherein the user interface element described above is applied to a plurality of ultrasound scans corresponding to cross sectional views of a bladder. A plurality of cross sectional area values are thus calculated. The volume of the bladder is then calculated by an algorithm which makes use of the plurality of cross sectional area values.

In an embodiment, the user interface element is applied to two ultrasound scans taken approximately orthogonally. These will preferably be a transverse and a sagittal bladder scan.

In a further embodiment the invention may be said to lie in a handheld ultrasound scan apparatus having a user interface including a touchscreen displaying elements able to be manipulated by a user via the touchscreen wherein the user interface includes the user interface element described above. In preference, the handheld ultrasound device has a weight of less than 500 grams.

Power consumption also contributes to portability. Lower power consumption means that a device can be battery powered and less reliant on frequent recharging.

In preference, the handheld ultrasound device is battery powered.

### BRIEF DESCRIPTION OF THE DRAWINGS

FIG. 1 is a handheld ultrasound device adapted to implement the method of the invention.

FIG. 2(a-f) show the application of the invention to a bladder image.

FIG. 3(a-l) show the application of the invention to the calculation of a bladder volume from two images.

FIG. 4 is a flow chart of the steps of a preferred embodiment of the invention.

FIG. 5 shows a diagrammatic representation of the application of the anisotropic filter of the invention.

FIG. 6 shows N equally spaced radii projected from a seed point over an ultrasound scan image.

FIG. 7 shows the image resulting from the application of an edge detection filter to an ultrasound scan image.

FIG. 8 is a diagrammatic representation of the operation of the PDAF of the invention.

FIG. 9 is a flow diagram of the steps of the PDAF of the invention.

FIG. 10 shows the bladder wall of the image of FIG. 3 delineated by perimeter line.

FIG. 11 shows a diagrammatic representation of the determination of the area of a cross section of a bladder image.

### BEST MODE FOR CARRYING OUT THE INVENTION

At times it is clinically useful to know the area or spatial volume of a structure within the body, in particular the volume of the bladder. Clinically, the volume of a patient's bladder may be important. A useful, non-invasive method of determining bladder volume is from one or more ultrasound images.

These images may be produced by any convenient means, however it is useful for these to be made by an inexpensive hand held ultrasound machine. This greatly expands the usefulness of the determination of bladder volume, since such a machine may be available in contexts such as nursing home use, or use by medical staff making home visits where a full size machine cannot economically be provided.

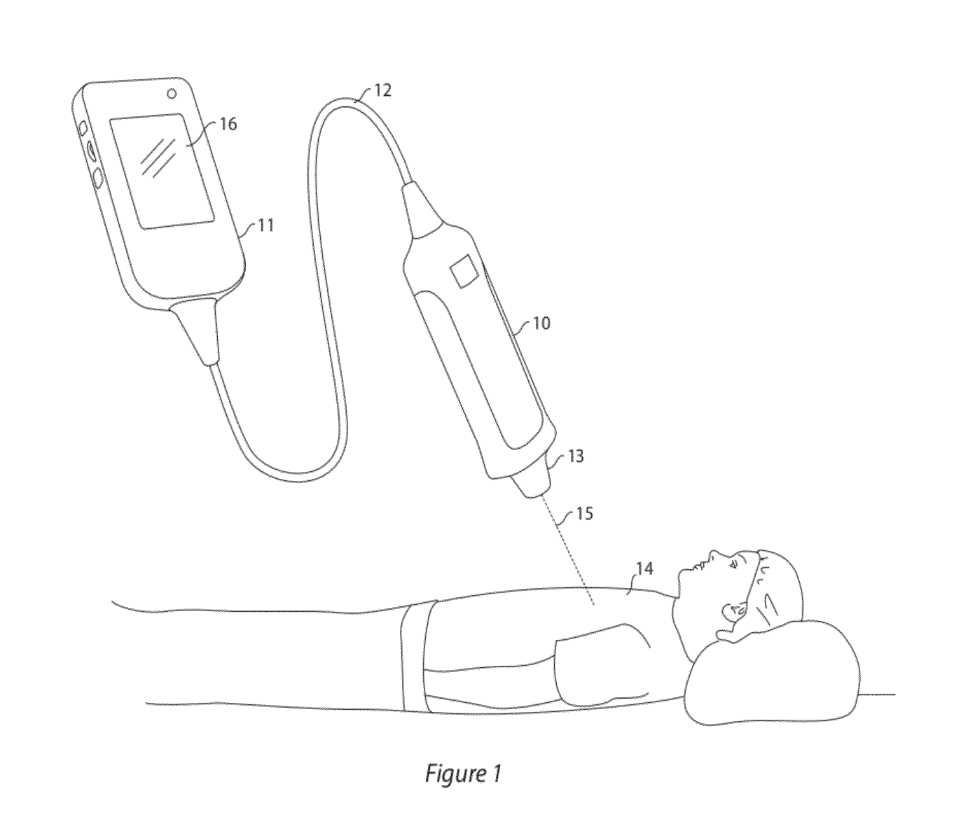

A useful machine is illustrated in FIG. 1. There is shown a hand held ultrasound scan device with an ultrasonic probe unit 102, a display and processing unit (DPU) 100 having a body 101 which is sized to be conveniently held in a user's hand. The DPU includes a display screen 104 and a cable 103 connecting the probe unit 102 to the DPU 100. The display screen 104 is a touch screen, which is used with a graphical user interface to control the device. The DPU may also include physical controls 106 and there may be physical controls 105 provided on the probe unit.

The probe unit 102 includes an ultrasonic transducer 108 which transmits pulsed ultrasonic signals through an acoustic window 107 into the body of a patient and receives returned echoes from the body of the patient. The ultrasonic transducer may be of any convenient type which allows for the production of a B-mode ultrasonic image.

In a preferred embodiment, the ultrasound device has a weight of less than 500 grams. For enhanced portability, it is preferably battery powered.

The ultrasound device may be a general purpose ultrasound scan device. A user interface is provided which allows this unit to be readily used to determine the volume of urine in a patient's bladder.

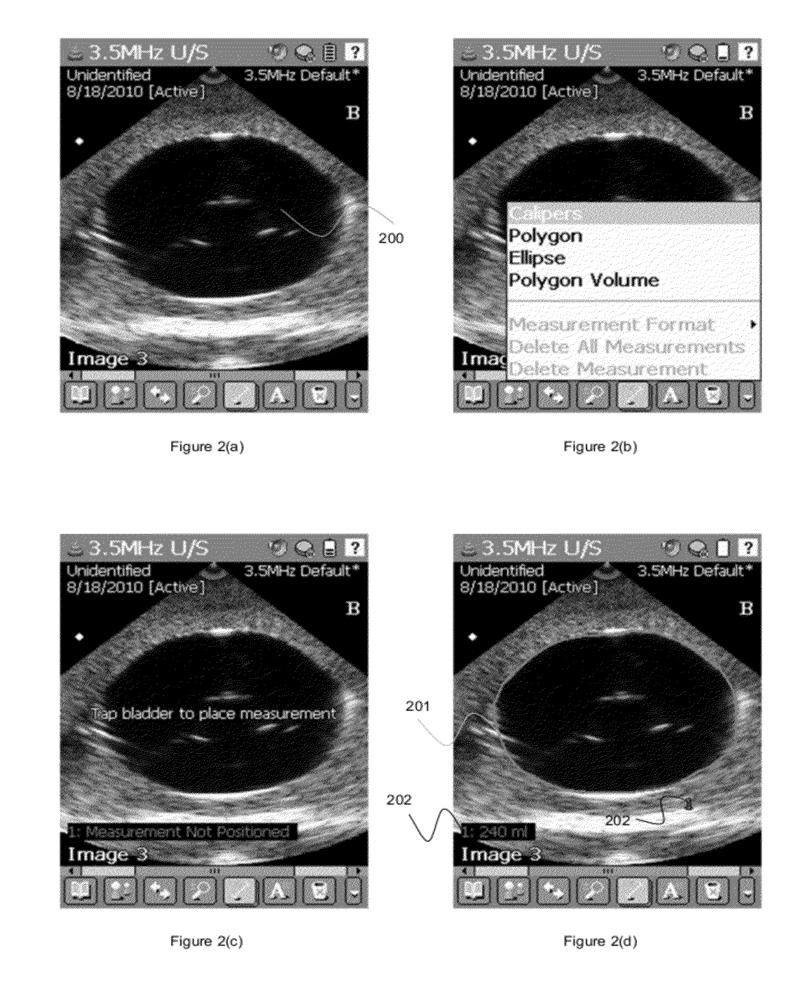

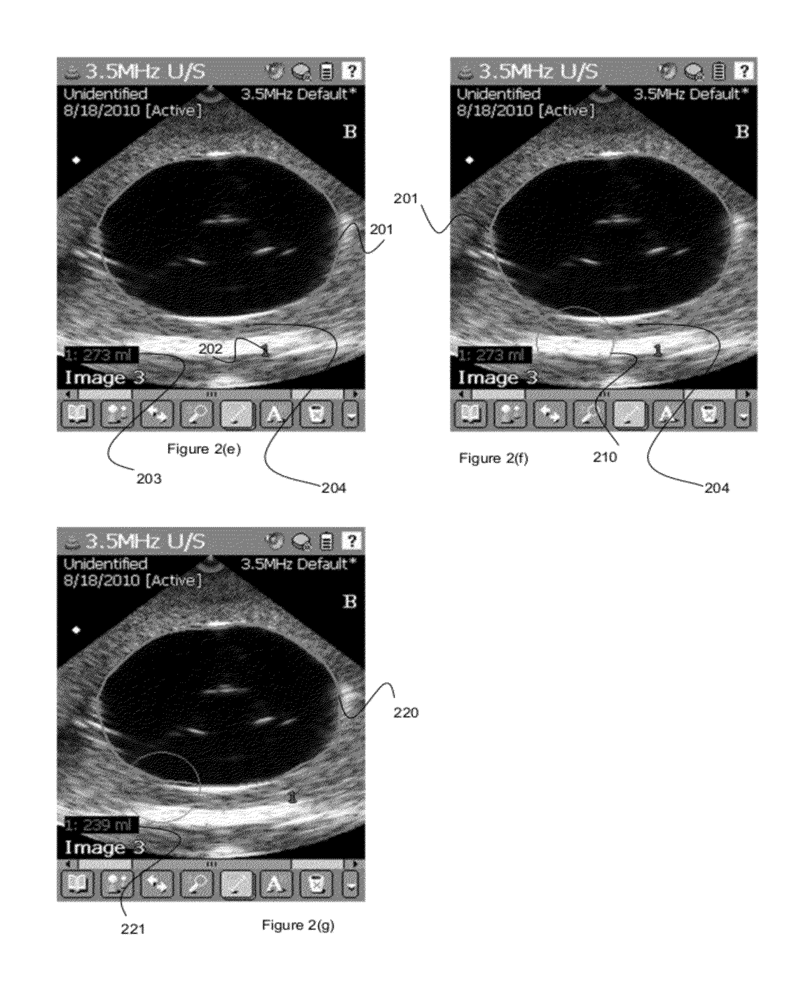

In order to determine the volume of urine in the bladder, an abdominal ultrasound scan is taken, as shown in FIG. 2(a). The bladder shows as an anechoic region 200 with a perimeter defined by reflections from the bladder wall, further surrounded by reflections from surrounding organs. The reflections show the classic “speckled” appearance which is the artifactual noise called speckle. Any reflectors within the bladder void are reverberation artifacts or random noise. This is a cross sectional view of the bladder, normally taken transversely across the patient's body.

In order to determine the bladder volume the user interface provides a specialised tag. A tag may be defined as any user interface element which may be attached to a scan image which carries information about the scan or the image or a portion of either. The information may be from any source, for example, inherent to the tag, acquired from the image, acquired from the process of placement of the tag or calculated within the tag. To use this tag, the user opens a menu as shown in FIG. 2(b), and selects the specialised tag “Polygon Volume”.

In order to define the perimeter of the bladder, it is necessary to establish a point which is definitely inside the perimeter. This point will be a seed point for later calculations by the tag. This may be done in a variety of ways, but the simplest manner is to have a user identify a point within the bladder.

Accordingly the user interface displays the screen of FIG. 2(c), where the user is invited to indicate a location which is within the bladder by tapping on the screen.

The tag is then placed. The tag calculates which points on the displayed image form part of the bladder perimeter by reference to the increased ultrasound reflectivity of that perimeter compared to the area within which the seed point was placed. The tag may or may not display an indicator indicating where the seed point was placed. A closed shape 201 is drawn on the image showing the calculated perimeter. This closed shape is identified by a tag identifier 202. The perimeter defines an area which is a cross section of the bladder. The volume of the bladder for the calculated perimeter is calculated and the result 203 is displayed.

The calculated perimeter 203 may not be coincident with the apparent bladder perimeter as displayed in the scan image and observed by an operator. Areas may be incorrectly included in or excluded from the defined cross-section.

These errors may be obvious to an operator. As can be seen in FIG. 2(e), an incorrectly included area 204 has been included in the cross section which is most likely not part of the bladder.

The interface provides a method for the user to adjust the calculated perimeter to more closely follow the apparent outline of the bladder in the scanned image. The adjusted perimeter will still be of a smooth shape, since the true outline of a bladder would not include sharp changes of angle.

To make such an adjustment, the user touches the screen on or close to, the drawn perimeter 201. This selects a point on the perimeter. The selected point may be marked on the image by an indicator such as a cross The user will then drag this point onto the apparent outline of the bladder. In order to preserve smoothness, all the points on the calculated perimeter, within a short distance, called the adjust radius, of the selected point, will also move. However, the amount of movement applied to the points to be moved varies according to distance from the selected point. The selected point moves the full distance by which that point is dragged by the user. Points on the adjust radius do not move at all. The adjust radius defines a portion of the perimeter which is able to be deformed such that the selected point is moved as directed by the user, the points on the perimeter at the adjust radius from the selected point do not move, and the perimeter remains continuous. The amount of movement of the other points within the adjust radius is calculated using a quadratic interpolation.

In an embodiment, the size of the adjust radius may be changed, either by user input, or algorithmically based on the characteristics of the scan image. The size of the adjust radius may be adjusted algorithmically based on the curvature or other properties of the perimeter in the vicinity of the selected point.

In an embodiment, the amount of movement of the points within the adjust radius may be determined by interpolation algorithms other than quadratic interpolation.

In order to assist the user in repositioning the perimeter, the adjust radius is shown on the image. This is illustrated in FIG. 2(f) where the adjust radius 210 is shown, surrounding a selected point within the incorrectly included area 204.

The user, in one or more steps, adjusts the perimeter to align with the apparent bladder volume in the scanned image. This leads to the display shown in FIG. 2(g) where the adjusted perimeter 220 now follows the apparent outline of the bladder. The calculated volume of the bladder 221 has been automatically adjusted.

Bladder shape and position can drastically vary with age, gender, filling degree and disease. The bladder shape is complex and cannot be represented exactly by a single geometrical formula such as ellipsoid, sphere etc. This explains the large error that several studies obtained when a single geometric model was used.

Where the bladder shape is irregular, which will usually be the case for a relatively empty bladder, greater accuracy in volume determination can be achieved by taking a further cross sectional scan of the bladder and combining the two area calculations in determining the bladder volume.

The user interface provides an assisted sequence—a series of instructions to the user, combined with specific controlled responses by the device.

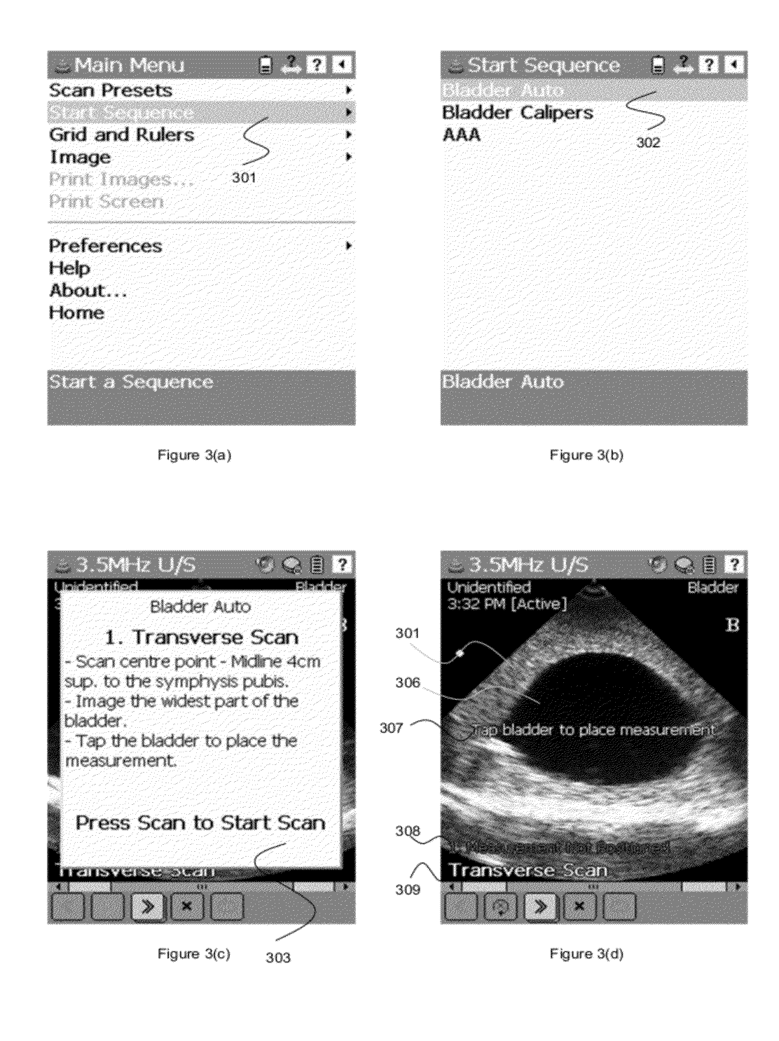

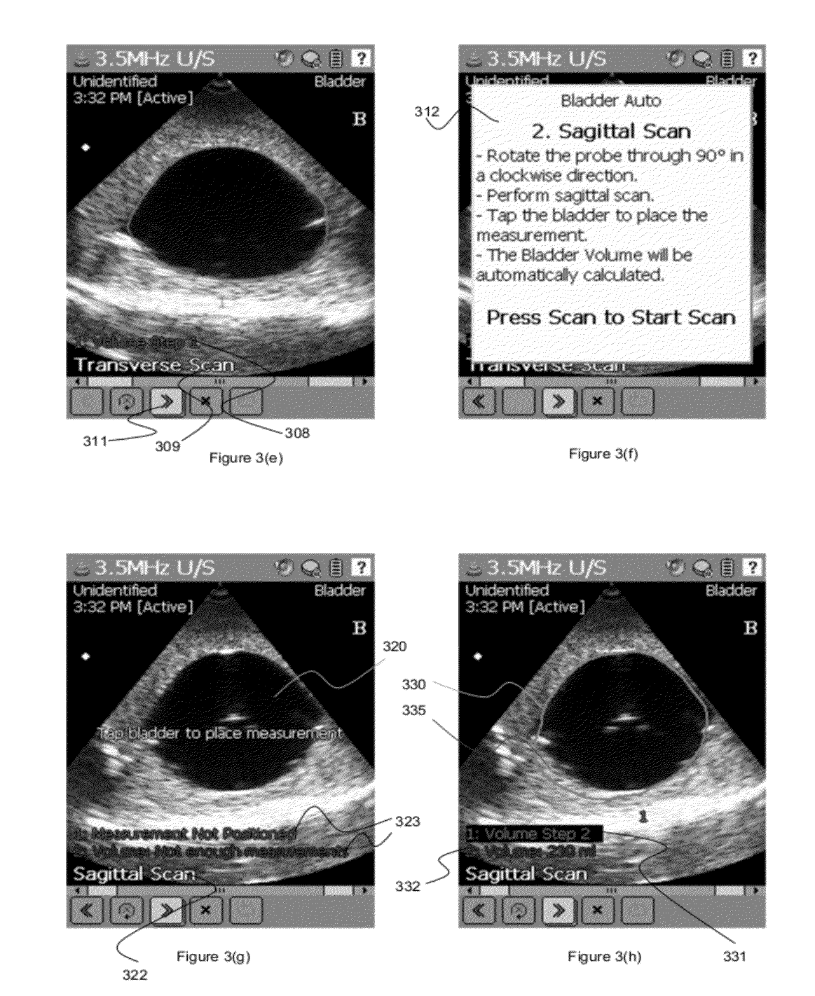

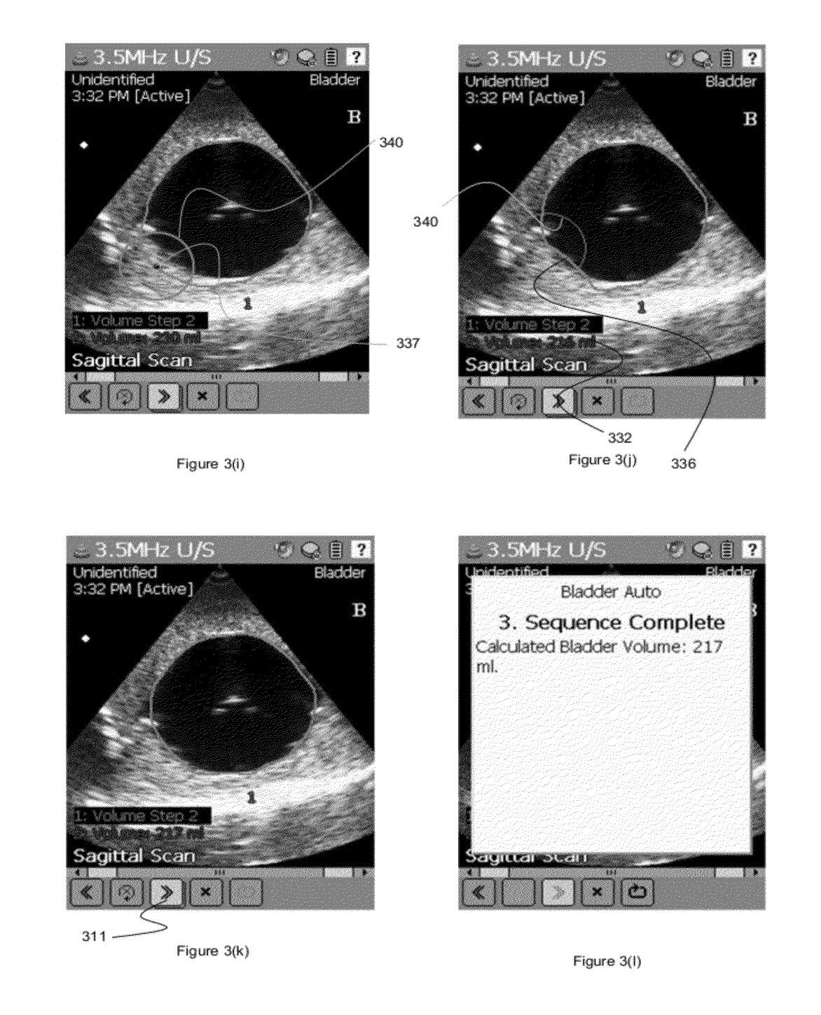

In order to determine a bladder volume, the user activates the main menu, as shown in FIG. 3(a), and selects the option “Start Sequence” 301. The screen of FIG. 3(b) is displayed. In this case the sequence is named “Bladder Auto” and the user selects this option 302. The first instruction screen 303, as illustrated in FIG. 3(c) is then shown. This instructs the user to obtain the first of the two scans, being a scan in the transverse direction. Instructions given may be operational, such as that the widest part of the bladder should be imaged or that the bladder image is to be tapped to continue the sequence. Instructions may also be clinical, such as a reminder as to where the widest part of the bladder is likely to be located.

The user then performs the scan. The result is the display of FIG. 3(d). The B-mode scan 305 shows the distinctive anechoic area 306 which indicates the bladder. An instruction line 307 instructs the user to tap within the area which the user can identify as the bladder. There is an information tag 308 indicating that the first measurement tag has not been placed. The title 309 indicates that this scan will be taken as the transverse scan.

The user then taps the screen to indicate the location of the bladder. This results in the display of FIG. 3(e). Here it can be seen that the bladder perimeter 310 has been determined and displayed. This determination is done using the specialised tag of the description of FIG. 2. The information tag 308 has been updated to indicate that that the first step of the sequence to determine the bladder volume is complete.

The user may now reposition the bladder perimeter, as described for FIG. 2, if the user believes that the automatic placing on the bladder perimeter line is not optimum. As described above, the user may drag sections of the perimeter to more closely match the apparent outline of the bladder.

When the user is satisfied with the placement of the bladder perimeter, the “continue” icon 311 is selected. The screen of FIG. 3(f) is then presented.

This is the second instructional screen 312. It instructs the user to turn the ultrasound probe ninety degrees clockwise and to perform a further scan. This is the sagittal scan. The user is further instructed to tap the area of the bladder on the sagittal scan in order for the volume calculation to be completed. Further operational or clinical instructions or advice may be included.

The probe unit is then turned ninety degrees and a further image is made. This results in the display of FIG. 3(g) which is a sagittal cross section of a bladder. The anechoic region 320 which corresponds to the bladder can be seen. The identifier tags 323 have been updated to indicate that the measurement tag has not been positioned and that there are not yet sufficient measurements available to calculate a volume, that is, the volume calculation will not be made until the bladder perimeter calculated from the sagittal scan is available. The title 322 indicates that this scan will be taken as the sagittal scan.

The user then taps within the area of the displayed bladder. The perimeter 330 of the bladder is then calculated and displayed as shown in FIG. 3(h). This is done using the specialised tag described by FIG. 2. The volume is calculated using information from both scans and this volume value 332 is displayed. The information tag 331 is updated to show that the second step of the sequence to determine the bladder volume is complete.

The automatic bladder perimeter placement may not perfectly correspond to the apparent bladder outline as shown on the scan. In the illustrated case, it can be seen that an incorrectly included area 335 is included within the bladder perimeter. An area may also be incorrectly excluded. The user is able to drag the perimeter to correct this.

To adjust the perimeter, the user selects a point on the perimeter, within the incorrectly included or excluded area. As shown in FIG. 3(i) the selected point 337 may be indicated by an indicator marker. The selected point is at the centre of an adjust radius 340.

As shown in FIG. 3(j), a user is able to drag a section of the perimeter 336 within an adjust radius 340 to align the displayed perimeter with the apparent bladder outline. The calculated volume 332 is updated as the perimeter is dragged.

When the user is satisfied that the displayed bladder perimeter matches the apparent bladder outline on the scan, as shown in FIG. 3(k), the user selects the “continue” icon 311. The final screen 350 of the sequence is displayed as shown in FIG. 3(l). This shows the final calculated volume.

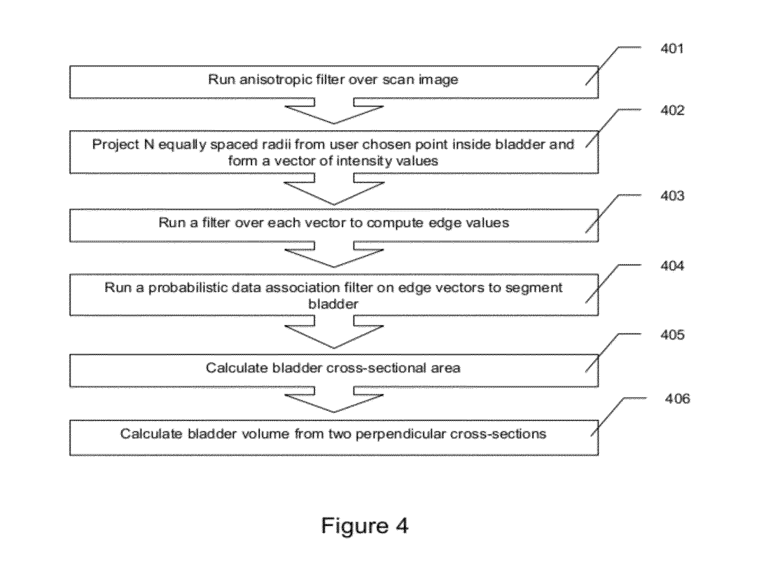

The images taken at each scan step are processed to display the bladder perimeter, after the user has indicated a seed point within the bladder. The steps of this process are shown in flow chart form in FIG. 4.

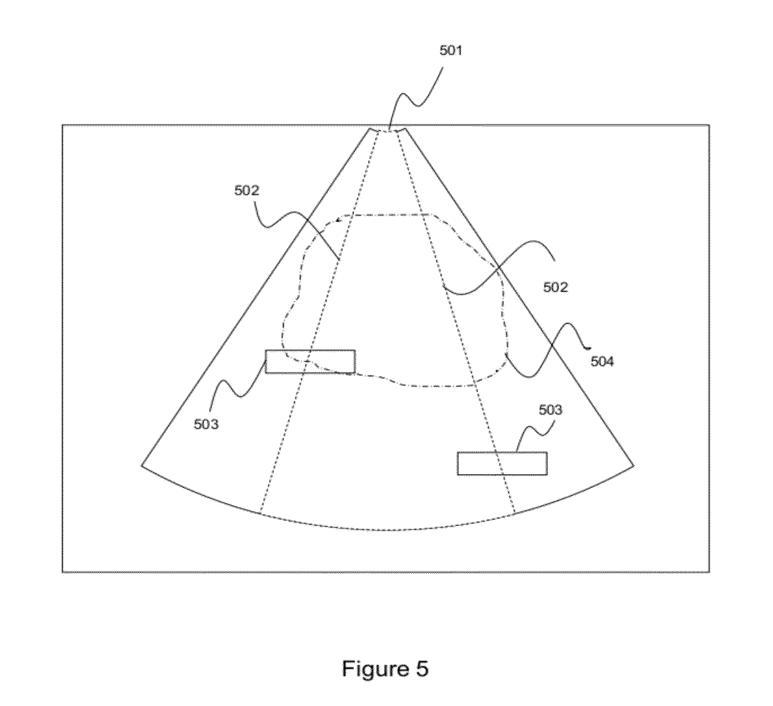

The image is first filtered to filter out noise, smooth speckle beyond the bladder edge and preserve the bladder boundary. In this first step 401, an anisotropic median filter is applied to the image data. This is shown diagrammatically in FIG. 5. A filter mask 503 is used which is a rectangle that is wider perpendicular to the scan line 502. The scan line for any image may be recreated as a line between any data point in the image and the source point 501.

This has the effect of filtering out noise more than filtering out speckle due to the speckle being arranged in bands that are perpendicular to the scan line.

In practice, this calculation may be undertaken after the image has been processed for display in a rectangular pixel grid. In this case, the filter mask may be a rectangle with sides which are aligned to the pixel grid. Since the angle by which the scanlines vary from alignment to the pixel grid is small, such a filter mask will also act to filter out noise to a greater extent than speckle.

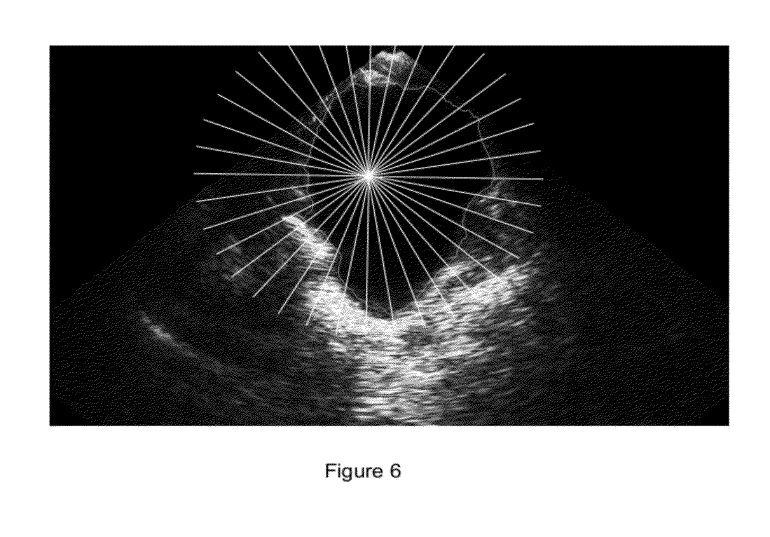

The point within the bladder nominated by the user is the seed location on the image. In the second step 402 N equally spaced radii are projected from the seed point as shown in FIG. 6.

The pixel intensities along each radius are extracted and stored. For the third step 403, an edge detector filter is applied along each radius.

This edge detector filter is a differential spatial filter used to calculate the edge value along each radius as shown in the equation below.

E(r)=(1−I(r))⁴{+I(r+2Δr)+I(r+Δr)+I(r)−I(r−Δr)−I(r−2Δr)−I(r−3Δr)}/3

Where E is the edge value, I is the pixel grey level value, and r is the distance from the seed point. Other filter equations may be used.

In this application, only the transition from dark regions to bright regions are significant since the edge detection algorithm starts from a point within the bladder area and it is known that the bladder is the darkest region within the image, hence only positive edge values are used in the algorithm.

Further, this means that transitions of brightness from very dark (anechoic) regions to lighter (echoic) regions are more likely to be part of the perimeter than transitions of brightness of the same magnitude where the transition is from an area of some echo to an area of greater echo. The latter type of transition is much more likely to be speckle.

Accordingly, as can be seen in the above equation, the edge detection filter includes a (1−I(r))⁴ term. Where the grey level of the pixel is low, meaning the pixel is in a substantially anechoic region, this term will be close to one. As the grey level increases, it will fall rapidly toward zero. This term therefore applies a heavy weighting to transitions starting from a very dark pixel, increasing the likelihood that these will be seen as part of the perimeter of the bladder.

In embodiments where the perimeter of a structure which is not so highly anechoic is being defined, the order of this term is reduced.

In embodiments where the structure being outlined is highly echoic and the background less echoic, the sign of the weighting is reversed, the term becoming I(r)⁴. Again, where the structure being outlined is less highly echoic, the order of the term is reduced.

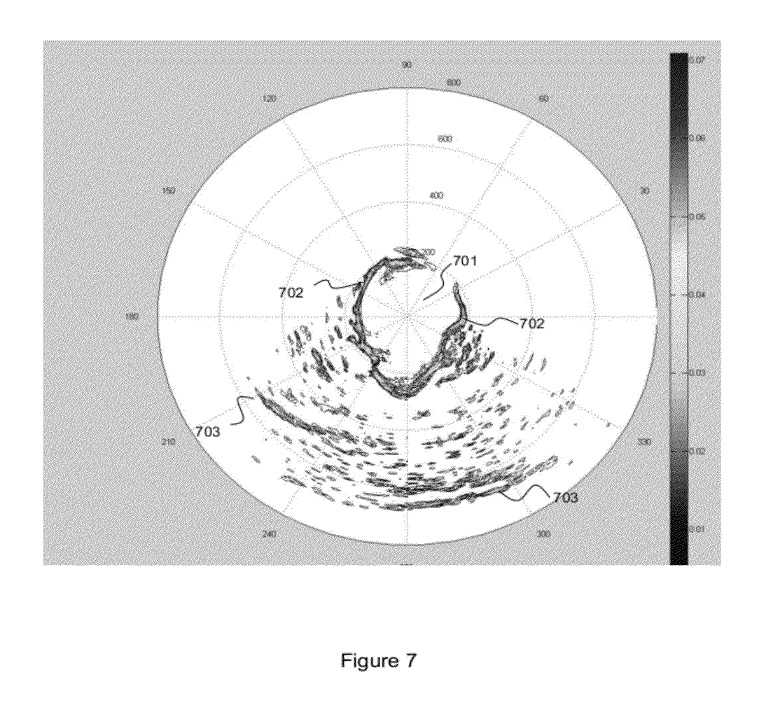

The image resulting from the application of this filter to a bladder scan image is shown in FIG. 7. It can be seen that there now exists a clearly visible anechoic area 701 which is substantially free of noise. The area 701 is surrounded by a reasonably clearly delineated echo producing perimeter 702. There are also other echo producing areas 703 which are not part of the perimeter of the bladder region.

The perimeter of the bladder is now able to be defined. This is described as segmenting the bladder image. This is done by applying probabilistic data association filter (PDAF) to the results of the edge detection step.

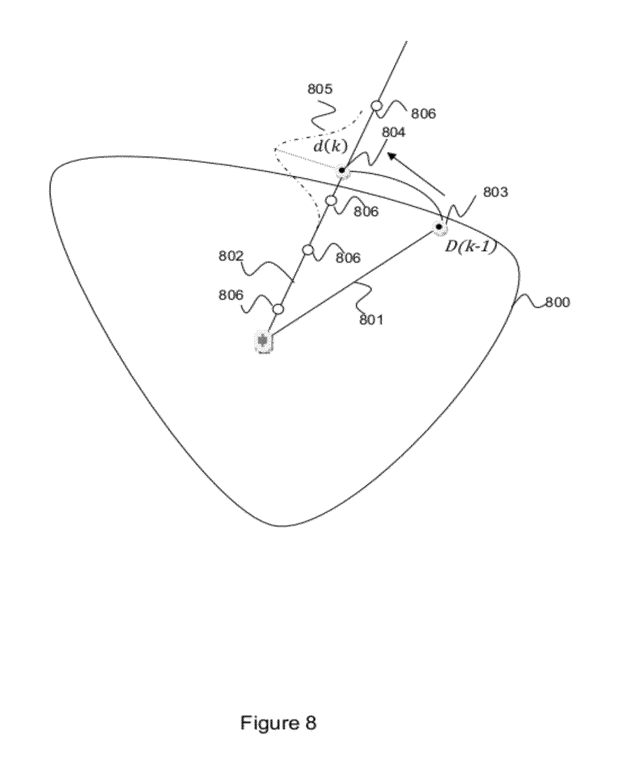

The basic operation of the PDAF is shown FIG. 8. There is the true bladder wall contour 800, which is the actual, physical bladder wall. The location of this wall is unknown. There are two of the N radii 801,802 projected at step 2 of the method 402. An estimate of the distance d(k) 804 of the bladder wall 800 from the seed point 807 along the k^{th} radius is made. The initial estimate is based on a “model” of the bladder which assumes that it is a circle centred on the seed point, hence the initial estimate is that the distance along each radius to the bladder wall is constant. Hence:

d(k)=D(k−1)

where D(k−1) 803 is the true value of the distance along the k−1^{th} radius to the bladder wall.

Each point rᵢ 806 along the radius is assessed for inclusion on the boundary. This is done using a formula which weights the likelihood of the point being on the boundary by both the magnitude of the edge E(r) and by its proximity to the model estimated boundary position d(k) 804. The proximity weighting is made by applying a weighting curve 805 which is a normal distribution with a mean d(k). The output of this step is a measurement estimate of the boundary radius Z(k).

A Kalman filter is then applied which weights the model estimate d(k) against the measurement estimate Z(k).

The output of this is an estimate for the value of the distance D(k) from the seed value to the true bladder wall.

The algorithm is applied sequentially to each radius sweeping around the perimeter twice to ensure that the perimeter estimate converges to a closed shape.

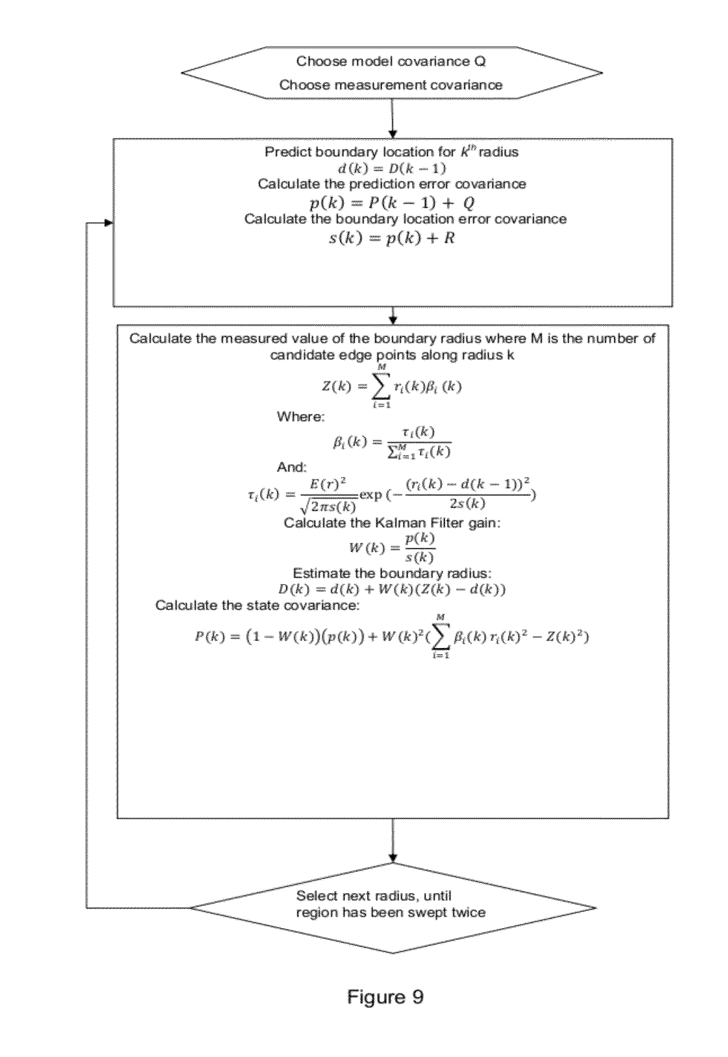

FIG. 9 shows the algorithm steps of the PDAF in greater mathematical detail.

The algorithm has proved to be very robust in segmenting the bladder contour in bladders of different shapes and sizes. Furthermore, the algorithm is able to accurately approximate the wall trajectory in the case of very large shadowing.

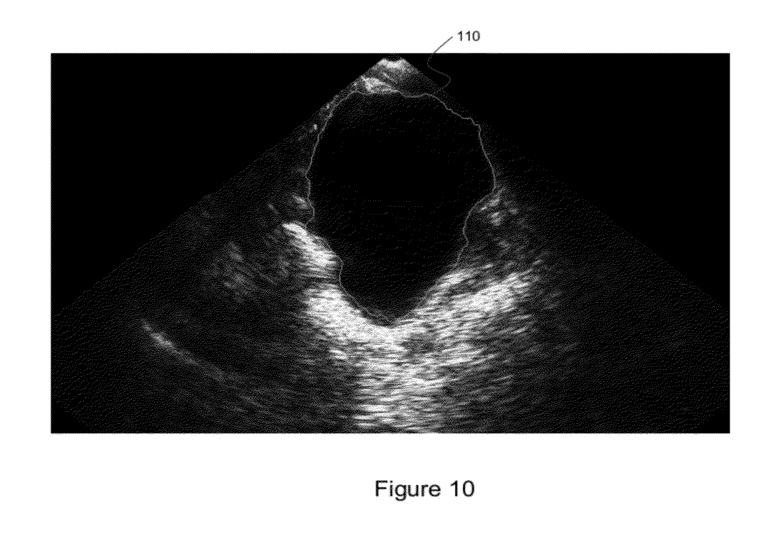

FIG. 10 shows the bladder wall of the image of FIG. 6 delineated by perimeter line 110.

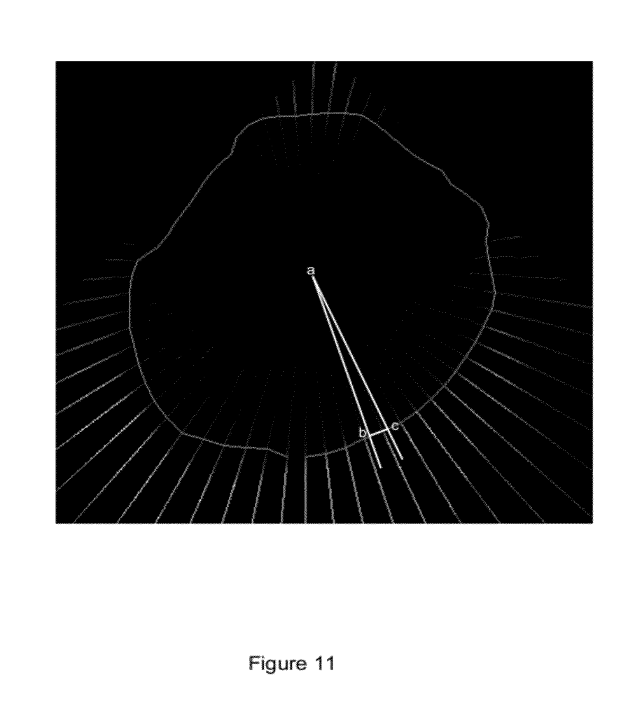

The cross sectional area of the bladder as shown in each of these images may be calculated by dividing the bladder into triangular elements made by the equally spaced radii from the seed points. This gives a series of triangles with vertices a, b, c of the type shown in FIG. 11.

The area A of each triangle may be calculated using:

$$A = \frac{1}{2} det ( \begin{matrix}{x}_{a} & {x}_{b} & {x}_{c} \\ {y}_{a} & {y}_{b} & {y}_{c} \\ 1 & 1 & 1\end{matrix} )$$

Summing the areas of each of the series of triangles will give the area of the cross section of the bladder.

In the case of the use of the specialized tag to calculate volume from a single scan, as described in relation to FIG. 2, the volume of the bladder is calculated assuming that the polygon is a circle and calculating a radius.

r=(Area/π)^{1/2}

Then calculating a volume from the radius assuming a sphere.

Volume=4πr³/3

This leads to an initial approximation of,

Volume=(4/(3√π))Area^{1.5˜}0.75 Area^{1.5}

Bladder calculations often use an ellipsoid approximation based on the three axis ‘diameters’ d₁, d₂ and d₃,

Volume=(π/6)d₁d₂d₃˜0.52d₁d₂d₃

However it is known in the art that because of the geometry of bladders it has been determined by empirical methods that more accurate results are obtained if the 0.52 scaling factor is replaced by 0.72.

Volume˜(0.72/0.52)*0.75 Area^{1.5}=1.04 Area^{1.5}

When both scan are available, as described by FIG. 3, these steps are applied to each of the transverse and sagittal bladder scans. The volume may then be calculated more accurately using both areas. One way of doing so is to use the following equation to determine the bladder volume V:

$$V = ∝ β {H}_{sagittal} {D}_{sagittal} {W}_{transverse}$$

$$Where \text{:}$$

$$α = \frac{{A}_{sagittal}}{{H}_{sagittal} {D}_{sagittal}}$$

$$β = \frac{{A}_{transverse}}{{W}_{transverse} {D}_{transverse}}$$

$$Hence \text{:}$$

$$V = \frac{{A}_{sagittal} {A}_{transverse}}{{D}_{transverse}}$$

where A_{sagittal} is the area of the bladder cross section area determined from the sagittal scan, Aₜᵣₐₙₛᵥₑᵣₛₑ is the area of the bladder cross section area determined from the transverse scan, and Dₜᵣₐₙₛᵥₑᵣₛₑ is the depth of the bladder as determined from the transverse scan.

In further embodiments, areas of the image which are definitely part of the bladder or definitely not part of the bladder may be separately identified. This may be done by any means, including direct user classification. Preferably, automated methods are used, such as filters which detect the characteristic streaky appearance of prostate tissue. The calculated parameter is checked to ensure that explicitly classified areas fall correctly within or outside of the bladder perimeter, and the perimeter adjusted if necessary.

The probe unit may also include an orientation sensor, which may be a gyroscope. In use, either a transverse or sagittal cross sectional image of the bladder is taken, preferably across the widest portion of the bladder. The face of the probe unit is then maintained in this position, while the probe unit is moved by the user through an arc approximately at right angles to the plane of the first acquired image. Images are taken whilst the probe is being moved, to give a series of 2D slices through the bladder.

Data from the gyroscope is used to track the relative positions of these slices. The location of the perimeter and the cross sectional area of the bladder are then determined in the 2D slices using the methods disclosed herein, while trilinear interpolation techniques are used to perform 3D scan conversion and estimate the volume of the bladder from the 2D slices.

Although the invention has been herein shown and described in what is conceived to be the most practical and preferred embodiment, it is recognised that departures can be made within the scope of the invention, which is not to be limited to the details described herein but is to be accorded the full scope of the appended claims so as to embrace any and all equivalent devices and apparatus.

1. A user interface element for a diagnostic ultrasound system adapted to be applied to an ultrasound B-mode image displayed to a user, the interface element including:
a seed point positioned by the user on the displayed image;
a perimeter defining a closed region of similar echoic intensity surrounding the seed point, the position of said perimeter being automatically calculated in response to the user positioning the seed point, where said region corresponds to a cross section through a physical feature within a body;
a first calculated value being an area of said closed region;
a second calculated value being a volume of said physical feature calculated using preselected assumptions about the shape of the physical feature wherein the user is able to modify the perimeter placement by dragging a selected point on the perimeter to a different location, the perimeter remaining continuous.

2. The element of claim 1 wherein there is a deformable portion of the perimeter being that portion of the perimeter within a selected radius of the selected point on the perimeter and the deformable portion deforms when moved such that points on the perimeter at the selected radius do not move, the selected point moves as dragged by the user using a graphical user interface and points on the perimeter within the deformable portion move by different amounts in such a manner that the perimeter remains continuous.

3. The element of claim 1 wherein the positions of the points on the deformable portion during and following the movement of the selected point are determined by a quadratic interpolation between the position of the selected point as dragged and the points on the perimeter at the selected radius.

4. The element of claim 2 wherein the deformation of the selected portion of the perimeter is displayed continuously as the user moves the selected point.

5. A handheld ultrasound scan apparatus having a user interface including a touchscreen adapted to display user interface elements able to be manipulated by a user via the touchscreen wherein the user interface includes the user interface element of claim 1.

6. The handheld apparatus of claim 5 wherein the apparatus has a weight of less than 500 grams.

7. A method of calculating the volume of a urinary bladder wherein the user interface element of claim 1 is applied to a plurality of ultrasound scans corresponding to spatially separated cross sectional views of said bladder to calculate a plurality of areas of closed regions, said areas of closed regions corresponding to areas of cross sections of the bladder at spatially separated planes, the volume of the bladder being calculated by an algorithm which makes use of the plurality of areas of cross section of the bladder.

8. The method of claim 8 wherein the user interface element is applied to two ultrasound scans taken approximately orthogonally.

9. A method of calculating the volume of a urinary bladder including the steps of acquiring a plurality of cross sectional scans of said bladder;
for each of said scans positioning a seed point upon a displayed image of that scan;
for each scan calculating the position of a perimeter defining a closed region of similar echoic intensity surrounding the seed point, the position of said perimeter being automatically calculated in response to the user positioning the seed point, where said region corresponds to a cross section through a physical feature within a body, wherein the user is able to modify the perimeter placement by dragging a selected point on the perimeter to a different location, the perimeter remaining continuous;
for each scan calculating a first calculated value being an area of said closed region, to give a plurality of areas of cross section of said bladder;
calculating the volume of said bladder using an algorithm which makes use of the plurality of areas of cross section of the bladder.
